(19) United States
(12) Patent Application Publication
(10) Pub. No.: US 2014/0336613 A1
(43) Pub. Date: Nov. 13, 2014

(54) INFUSION-TUBE CLAMP FOR AN INFUSION PUMP, AND A METHOD FOR USING SAID INFUSION-TUBE CLAMP

(71) Applicants: Stefan Roth, Melsungen (DE); Hans-Josef Gerlach, Marsberg (DE); Joerg Scherer, Aalen (DE); Willy Huwyler, Cham (CH); Marc Pfyl, Zug (CH)

(72) Inventors: Stefan Roth, Melsungen (DE); Hans-Josef Gerlach, Marsberg (DE); Joerg Scherer, Aalen (DE); Willy Huwyler, Cham (CH); Marc Pfyl, Zug (CH)

(73) Assignee: B. Braun Melsungen AG, Melsungen (DE)

(21) Appl. No.: 14/352,248

(22) PCT Filed: Aug. 7, 2012
(86) PCT No.: PCT/EP2012/065460

§ 371 Date: Jun. 10, 2014

(30) Foreign Application Priority Data
Oct. 18, 2011 (EP) 11185623.3

(51) Int. Cl. A61M 39/28

(52) U.S. Cl. CPC A61M 39/28; A61M 39/284
USPC 604/500; 604/250

(57) ABSTRACT

The invention relates to an infusion tube clamp for being inserted in an infusion pump comprising a base member having an infusion pump receiving area, a first clamping leg, and a second clamping leg for squeezing an infusion tube, wherein the two clamping legs are swivel-connected to each other at a first infusion tube clamp end. The infusion tube clamp also includes a click device for safely positioning the two clamping legs in more than one position relative to each other.

### CROSS REFERENCE TO RELATED APPLICATIONS

This application is the U.S. national phase application of PCT International Application No. PCT/EP2012/065460 filed Aug. 7, 2012, which claims priority to European Patent Application No. EP 11185623.3 filed Oct. 18, 2011, the contents of each application being incorporated by reference herein.

### FIELD OF THE INVENTION

The invention on the one hand relates to an infusion tube clamp for being inserted in an infusion pump comprising a base member having an infusion pump receiving area, comprising a first clamping leg and a second clamping leg, the two clamping legs being swivel-connected to each other at a first infusion tube clamp end.

On the other hand, the invention relates to a method of using an infusion tube clamp at an infusion pump in which a first clamping leg and a second clamping leg of the infusion tube clamp are swiveled toward each other around an infusion tube clamp end portion so that an infusion tube can be squeezed in this way.

Moreover, the invention relates to an infusion pump comprising a seat for an infusion tube clamp arranged on the tube side.

### BACKGROUND OF THE INVENTION

Especially generic tube-side snap or safety clamps, also referred to as FFC (free flow clamp), are known from prior art in various configurations. Within the scope of an infusion set they are frequently provided already at an infusion tube where they are intended to either enable or inhibit flow of an infusion fluid through the infusion tube. This relates both to a state before or after mounting in an appropriate infusion pump and to a state during mounting into the infusion pump. As a rule, the tube-side safety clamp or FFC is closed before and after mounting, while it can be automatically opened depending on the operating state of the infusion pump for example regarding an infusion administration. For inserting the infusion tube comprising the FFC almost always a pump flap of an infusion pump is opened so as to obtain access to an infusion tube guide and especially to an appropriate FFC seat in this way.

Generally such tube-side FFC is usually positively controlled by manual operation of a pump flap of an infusion pump and, respectively, is automatically closed upon opening or closing the pump flap so as to inhibit inadvertent flow of the infusion fluid through the infusion tube. It may be necessary in this case that the tube-side safety clamp first has to be manually closed before the pump flap can be correctly closed. However, frequently it is also the case that the tube-side FFC is closed already during insertion but is then opened again immediately upon closing the pump flap. A tube-side FFC closed after closing the pump flap can be automatically re-opened for pump operation by means of an actuator of the infusion pump. Apart from infusion pumps having such on-function or opening-function, there exist further infusion pumps having an additional actuator for a corresponding off-function or closing-function for closing the tube-side FFC. Moreover, infusion pumps which, apart from such a tube-side FFC, in addition have a device clamp with an actuating on- and off-function belong to prior art. Most tube-side FFC are still rather complex or an additional device clamp is required at an infusion pump so as to be able to ensure a reliable infusion fluid flow stop inside an infusion tube.

### SUMMARY OF THE INVENTION

It is an object of the invention to further develop especially generic infusion tube clamps so that both manufacture of an appropriate infusion set comprising an infusion tube and use of such infusion tube clamps at an infusion pump is rendered substantially easier and more reliable.

An object of the invention is achieved by an infusion tube clamp for being inserted in an infusion pump comprising a base member having an infusion pump receiving area, comprising a first clamping leg and a second clamping leg, wherein the two clamping legs are swivel-connected to each other at a first infusion tube clamp end, and wherein the infusion tube clamp excels by a click device for safely positioning the two clamping legs in more than one position relative to each other.

Advantageously the present infusion tube clamp can be reliably retained in at least two different snap-in positions by means of the click device.

In accordance with aspects of the invention, the term “click device” describes all devices by means of which the two clamping legs can be retained or positioned in different positions relative to each other.

Advantageously the click device includes a non-detachable detent element and a detachable detent element, wherein the at least one click element can pass the non-detachable detent element only once in a first direction and wherein the at least one click element can pass the detachable detent element both in the first direction and in a second direction opposed to the first direction.

The term “detachable detent element” describes a detent element which can be deflected vis-\grave{v}-vis the click element further than regarding the “non-detachable detent element”, as will be further illustrated hereinafter, so that the click element can pass the detachable detent element in two directions.

The two clamping legs are interconnected at the first infusion tube clamp end to be ideally inseparable. Advantageously, the present infusion tube clamp is manufactured as a one-piece injection molded part.

The present infusion tube damp is arranged on the tube side in accordance with the invention and in so far differs from device clamps installed on the infusion pump side. However, the present tube-side infusion tube clamp is suited for being inserted in an infusion pump and accordingly it includes an infusion pump receiving area by means of which the infusion tube clamp can be arranged in a corresponding infusion pump-side seat. Herein the present infusion tube clamp differs from other tube clamps.

So as to be advantageously employed in an infusion pump the infusion tube clamp preferably has an actuating flank for an infusion pump locking bar.

An infusion pump of this type preferably is a flexible tube pump. Since generic infusion pumps are well known from prior art, they will not be discussed in more detail hereinafter.

The infusion tube clamp advantageously has a manufacturing state in which the two clamping legs are preferably inseparably interconnected only on one side at the first infusion tube clamp end. On the side of the click device the two clamping legs are not yet interconnected so that an infusion tube can be inserted in the infusion tube clamp or in a squeezing means for the infusion tube by an extremely simple procedure. Accordingly, a more complicated threading of the infusion tube into the infusion tube clamp is dispensed with, whereby especially an infusion set can be manufactured and provided in a substantially easier way.

A preferred configuration variant provides that the click device includes a respective operating state having an opening position, especially of a squeeze means for squeezing the infusion tube, and having a squeezing position, especially of the squeeze means for squeezing the infusion tube, in which at least one click element of the click device can be arranged to be engaged in a first detent element in the opening position and in a second detent element in the squeezing position.

Furthermore, it is of advantage when the two detent elements are arranged to be connected in series at the second clamping leg. In this way especially the building height of the click device can be kept small. The term “in series” relates to the direction of movement of the two clamping legs in accordance with the invention.

Preferably, in terms of space one of the detent elements is always arranged more closely to the first clamping leg than the other one of the detent elements.

In the simplest case the squeeze means can be configured of two merely rod-shaped clamping legs. Preferably the squeeze means includes a squeeze edge at the first clamping leg and a squeeze edge support at the second clamping leg, wherein the squeeze means is still or again opened in a first snap-in position of the snap fit and being closed in at least one further snap-in position of the snap fit.

It is understood that the click device can have various configurations. For example, a first click element of the click device is provided at the first clamping leg and a corresponding detent element of the click device is provided at the second clamping leg, while a further click element of the click device is arranged at the second clamping leg and a corresponding further detent element of the click device is arranged at the first clamping leg.

The design of the infusion tube clamp can be realized in an especially simple and space-saving manner, however, when the click device includes at least one click element associated with the first clamping leg and at least two click elements associated with the second clamping leg for engaging the at least one click element.

Advantageously the click device is arranged at an area spaced apart from the first infusion tube clamp end so that between the click device and the first infusion tube clamp end sufficient space is retained for designing a reliably functioning squeezing and guiding of the infusion tube.

In terms of construction, the present infusion tube clamp can be further facilitated are arranged relative to one another so that when closing the click device the click element first engages in one of the two detent elements, before the click element can be engaged in the other one of the two detent elements.

Moreover, an object of the invention is also achieved by a method of using an infusion tube clamp at an infusion pump in which a first clamping leg and a second clamping leg of the infusion tube clamp are swiveled toward each other around an infusion tube clamp end portion so that an infusion tube can be squeezed in this way, wherein the method further excels by the fact that a click device is engaged by at least one click element with a first detent element into a first snap-in position in which the infusion tube is retained between the two clamping legs, and that the click device is engaged by the at least one click element or by another click element with a second detent element into a second snap-in position in which the infusion tube is squeezed.

Advantageously the click device is transferred to the first snap-in position as soon as the infusion tube is inserted into the infusion tube clamp, preferably transversely to the longitudinal extension of the two clamping legs. Thus the click device directly materializes an attachment hinge, as the infusion tube is radially surrounded on all sides by the infusion tube clamp. As a consequence, the infusion tube clamp cannot be removed from the infusion tube any more without destruction of the infusion tube or the infusion tube clamp itself.

In this respect, it is of advantage when the infusion tube clamp includes, at least in an operating state, a tube guide radially closed on all sides having a tube guide direction arranged transversely to the longitudinal extension of the two clamping legs.

Ideally the click device is transferred into the second snap-in position as soon as it is correctly inserted in the infusion pump. This excludes the risk of inadvertently supplying an infusion solution to a patient, if the latter is already connected to the infusion tube.

In a preferred method variant, the second detent element is preferably deflected for re-opening the infusion tube so that the click device returns from the second snap-in position into the first snap-in position.

Hence for opening a respective squeeze means the second detent element of the infusion tube clamp is deflected, whereby a click element engaged in the second detent element before is released.

For deflecting or releasing one of the two detent elements it is advantageous when one of the two detent elements has an actuating surface for an actuator of an infusion tube clamp actuation of the infusion pump, wherein especially the second one of the two detent elements is configured to be deflectable further than the first one of the two detent elements.

According to another aspect of the invention, the present object is also achieved by an infusion pump comprising a seat for an infusion tube clamp arranged on the tube side and comprising an actuator for actuating the tube-side infusion tube clamp which excels by an infusion tube clamp according to any one of the features described here, as the infusion pump can have a simpler design than it has been usual so far.

Especially it requires substantially merely an appropriately configured seat for accommodating the present infusion tube clamp and, respectively, a seat for the infusion pump receiving area of the infusion tube clamp.

Advantageously, on the basis of the present infusion tube clamp a new generation of infusion pumps as well as corresponding extension components and corresponding accessories can be provided.

The infusion tube clamp ideally is designed in one piece and has a non-detachable detent element and a detachable detent element. By manually closing or locking a pump flap of an infusion pump the infusion tube clamp is closed. When the detachable detent element is deflected, the infusion tube clamp changes from a closed to an open position. With the aid of a pump-side actuator the detachable detent element of the infusion tube clamp can be deflected only when the pump flap is closed. Then the infusion tube clamp opens: By manually unlocking the pump flap the infusion tube clamp is closed, before the pump flap is opened. The infusion tube clamp advantageously need no longer be threaded onto the passage or the infusion tube, but it is simply clipped on.

Moreover, due to the present invention substantially the following further advantages are resulting:
A free-flow protection is fully achieved via the tube-side (set-based) FFC.
No device-side FFC is required.
Upon closing and opening the manually locked pump flap the FFC is always closed via a mechanical positive control. Safety feature
For the pump operation the infusion pump opens the tube-side FFC by actuation.
Actuated closing is not required.
The infusion pump-side actuator only has an on-function.
It is ensured by the infusion pump itself that the infusion pump cannot be started without a correctly inserted FFC.-> Safety feature
The use of infusion sets without FFC is not possible.-> Safety feature
The tube-side FFC has a one-piece structure.
Easier assembly during production by clipping on instead of threading.
For protection against undesired manipulation the FFC cannot be removed without damaging the tube or the FFC itself, for example by rupture of the non-detachable detent element.-> Safety feature

### BRIEF DESCRIPTION OF THE DRAWINGS

The invention is best understood from the following detailed description when read in connection with the accompanying drawings. Included in the drawings are the following figures:

FIG. 1 schematically shows a view of a first embodiment of an infusion tube clamp including a click device comprising one single click element and two detent elements arranged at an infusion pump;

FIG. 2 schematically shows a view of the infusion tube clamp of FIG. 1 in a first operating state as regards a squeezing position;

FIG. 3 schematically shows a further view of the infusion tube clamp of FIGS. 1 and 2 in another operating state as regards an opening position;

FIG. 4 schematically shows a view of a second embodiment of a different infusion tube clamp in a manufacturing state;

FIG. 5 schematically shows another view of the infusion tube clamp of FIG. 4 in a first operating state as regards an opening position; and

FIG. 6 schematically shows a view of the infusion tube clamp of FIGS. 4 and 5 in another operating state as regards a squeezing position.

### DETAILED DESCRIPTION OF THE PREFERRED EMBODIMENTS

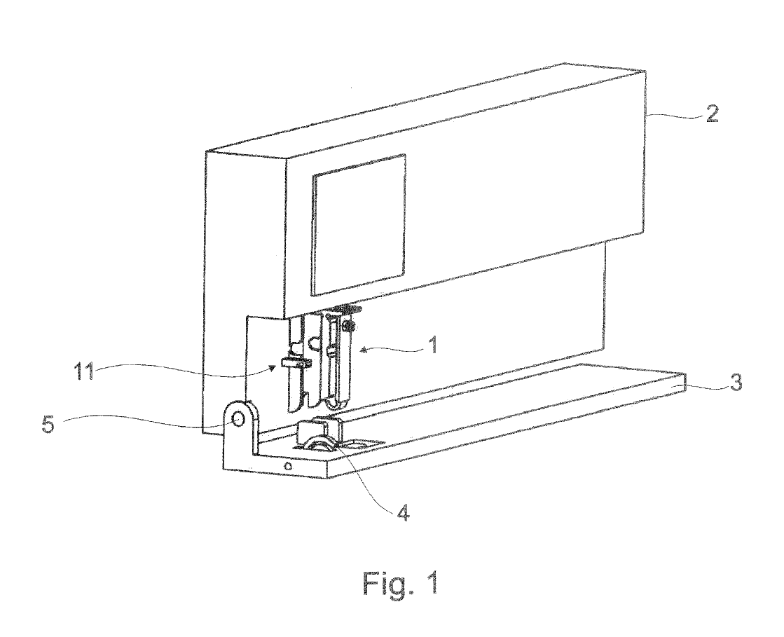

The infusion tube clamp 1 illustrated in FIG. 1 is placed in this embodiment to be vertically aligned in an infusion pump 2 having an opened pump flap 3. A locking bar 4 of the pump flap 3 is provided at the pump flap 3, the latter being pivoted about a rotary catch 5 of the infusion pump 2.

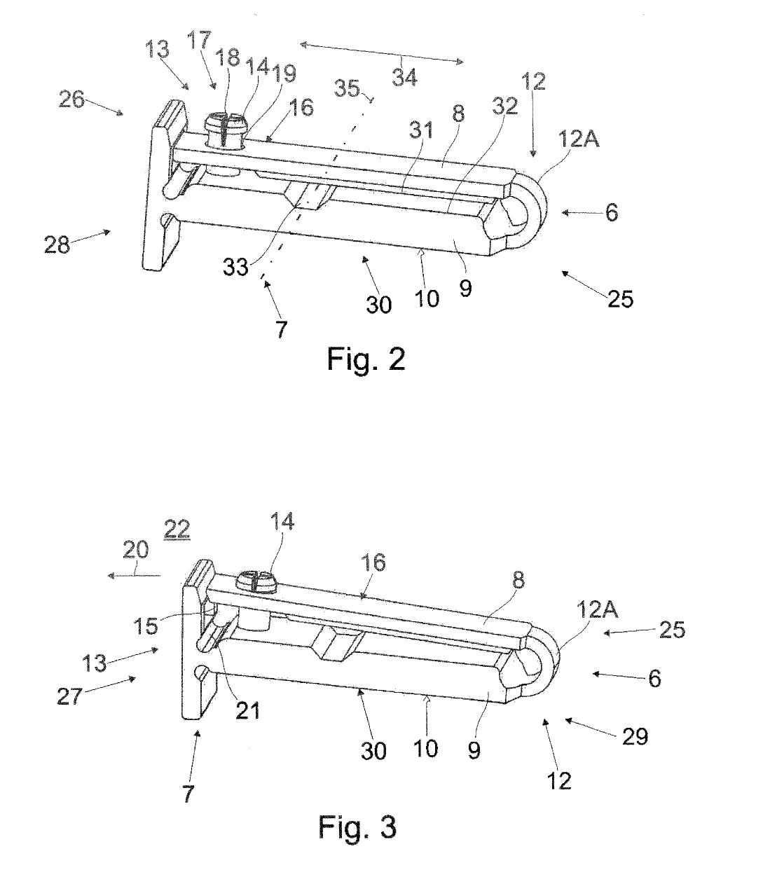

The infusion tube clamp 1 (cf. especially FIGS. 2 and 3) comprises a base member 6 having an infusion pump receiving area 7, comprising a first clamping leg 8 as well as a second clamping leg 9.

The infusion pump receiving area 7 is provided at the rear side 10 of the second clamping leg 9 so that the infusion tube clamp 1 can be attached in an appropriately configured seat 11 of the infusion pump 2 to be accurately fitting.

The infusion tube clamp 1 has a one-piece structure, as the two clamping legs 8 and 9 are interconnected inseparably but sliding at a first infusion tube clamp end 12 by means of a tab 12A.

The infusion tube clamp 1 further excels by a click device 13 for safely positioning the two clamping legs 8 and 9 at more than one position relative to each other, wherein for this purpose the click device 13 includes a non-detachable first detent element 14 and a detachable second detent element 15 (cf. FIG. 3) which can optionally be engaged in a click element 16 of the click device 13.

While the two detent elements 14 and 15 are configured at the second clamping leg 9, the click element 16 in this embodiment is realized directly by the first clamping leg 8.

The first detent element 14 is formed by an offset web 17 which extends perpendicularly from the second clamping leg 9 and has a slotted head area 18. In the area of the first detent element 14 the click element 16 includes a material recess 19 through which the offset web 17 is put so that the click element 16 is non-detachably engaged in the first detent element 14.

The second detent element 15 is a detent catch 21 deflectable in the deflecting direction 20 which is arranged at the head side 22 of the click element 16. When the two clamping legs 8 and 9 are moved further toward each other, the click element 16 can also pass the deflectable detent catch 21, wherein the infusion tube clamp 1 squeezes the infusion tube.

According to the representations in accordance with FIGS. 2 and 3, the infusion tube clamp 1 is provided in an operating state 25 as regards a squeezing position 26 (FIG. 2) and as regards an opening position 27 (FIG. 3).

In the opening position 27 the click element 16 according to a first snap-in position 28 of the two clamping legs 8 and 9 is engaged at the first detent element 14. The click element 16 abuts against the offset head area 18, wherein the two clamping legs 8 and 9 tend to move apart from each other by means of a spring force applied by the tab 12A.

In the squeezing position 26 the click element 16 according to a second snap-in position 29 of the two clamping legs 8 and 9 is engaged at the second detent element 15 so that an infusion tube not shown here is squeezed off by a squeeze means 30.

The squeeze means 30 forms a clamping edge 31 at the first clamping leg 8 and a clamping edge support 32 at the second clamping leg 9.

Moreover, the infusion tube clamp 1 includes a tube guiding space 33 radially closed on all sides which is substantially configured by the two clamping legs 8, 9, the tab 12A and the first detent element 14 of the click device 13. A tube guiding direction 35 extends transversely to the longitudinal extension 34 of the two clamping legs 8 and 9.

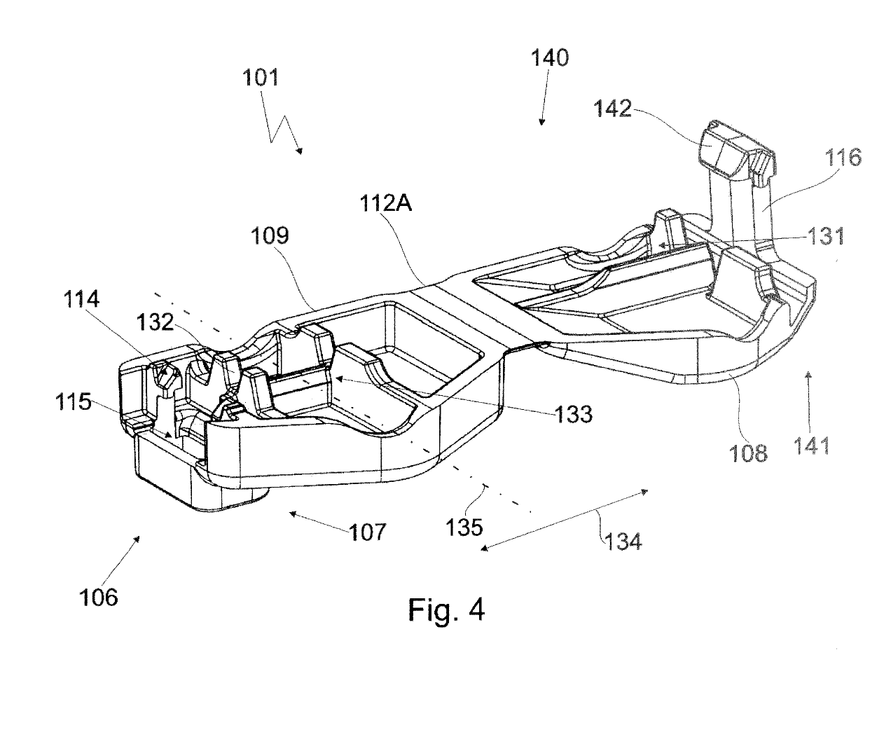

The further embodiment shown in FIGS. 4 to 6 of an infusion tube clamp 101 includes a base member 106 as injection molded part comprising a first clamping leg 108 and a second clamping leg 109 as well as an infusion pump receiving area 107.

The two clamping legs 108 and 109 are swivel-connected to each other in a fixed manner at a first infusion tube clamp end 112 by means of a resilient tab 112A.

According to the representation according to FIG. 4, the infusion tube clamp 101 is shown to be stretched in a manufacturing state 140 in which the click device 113 is not yet engaged in its click element 116 at either of the two detent elements 114 and 115. In this respect, an infusion tube (not shown here) can be easily inserted in the tube guiding chamber 133 of the still open infusion tube clamp 101 in the tube guiding direction 135 extending transversely to the longitudinal extension 134 of the two clamping legs 108 and 109.

The first detent element 114 is configured to be non-detachably engageable and the second detent element 115 is configured to be detachably engageable in the click device 116, wherein the first detent element 114 has an appropriately massive design so that it cannot be deflected or can be deflected only negligibly.

The click device 113 thus further includes, apart from the detent elements 114 and 115, a click element 116 adapted to correspond to the two detent elements 114 and 115. At the end 141 of the first clamping leg 108 the click element 116 projects approximately vertically therefrom and includes a snap catch 142 by means of which it can snap or engage in the respective detent element 114 and 115, respectively. The click element 116 in this case includes an at least sufficient elasticity so as to be adapted to engage more easily in the snap catch 142 at the respective detent element 114 and 115, resp.

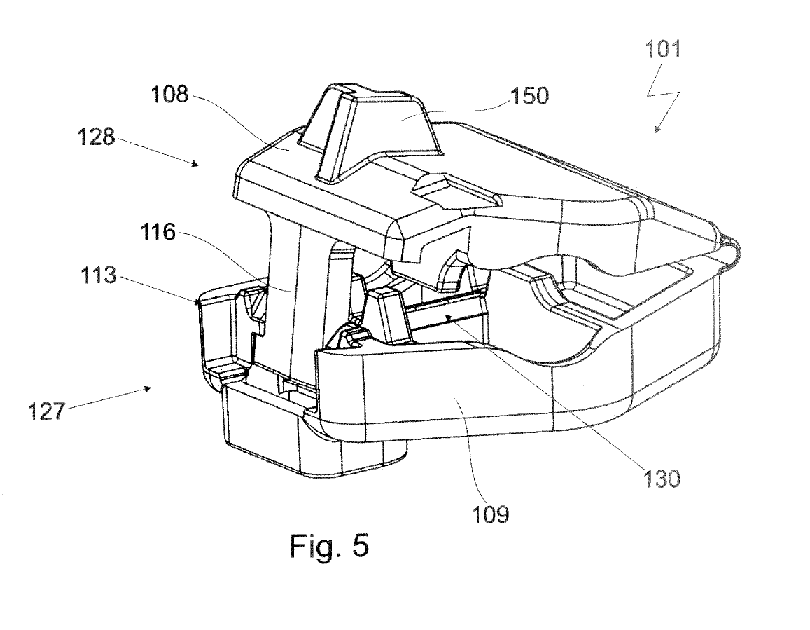

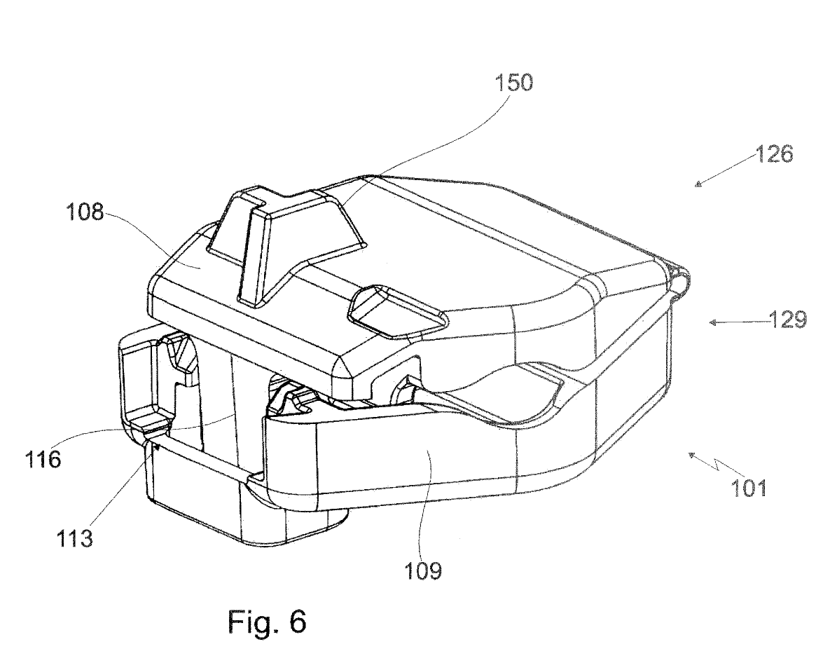

In accordance with the representations of FIGS. 5 and 6, the infusion tube clamp 101 is shown in an operating state 125.

In this case the infusion tube clamp 101 is in an opening position 127 (cf. FIG. 5) in which the click element 116 is engaged in the first detent element 114. The infusion tube clamp 101 in so far exhibits a first snap-in position 128.

If, however, the click element 116 is engaged in the second detent element 115, the infusion tube clamp 101 is provided in a squeezing position 126 (cf. FIG. 6) in which the infusion tube clamp 101 and, respectively, the two clamping legs 108 and 109 adopt a second snap-in position 129.

The infusion tube clamp 101 comprises a squeeze means 130 having a squeeze edge 131 and a squeeze edge support 132.

Moreover, at the first clamping leg 108 an actuating flank 150 for an infusion pump locking bar (cf. FIG. 1, reference numeral 4) is provided.

It is understood that the afore-illustrated embodiments are merely first configurations of the infusion tube clamp according to aspects of the invention. So far the configuration of the invention is not restricted to these embodiments.

All features disclosed in the application documents are claimed as being essential to the invention, if they are novel either individually or in combination vis-à-vis prior art.

1.-12. (canceled)

13. An infusion tube clamp for being inserted in an infusion pump, comprising:
a base member including an infusion pump receiving area, a first clamping leg and a second clamping leg for squeezing an infusion tube, wherein the two clamping legs are swivel-connected to each other at a first infusion tube clamp end; and
a click device for safely positioning the two clamping legs in more than one position relative to each other wherein the click device includes at least one click element, a non-detachable detent element and a detachable detent element wherein the non-detachable detent element can be passed by the at least one click element only once in a first direction and the detachable detent element can be passed by the at least one click element both in the first direction and in a second direction opposed to the first direction.

14. The infusion tube clamp according to claim 13, wherein the click device has an operating state including an opening position and a squeezing position in which at least one click element of the click device can be arranged to be engaged in the opening position in a first detent element and in the squeezing position in a second detent element.

15. The infusion tube clamp according to claim 13, wherein the two detent elements are connected in series.

16. The infusion tube clamp according to claim 13, wherein the at least one click element is arranged at the first clamping leg and the first and second detent elements are arranged at the second clamping leg for engaging the at least one click element.

17. The infusion tube clamp according to claim 14, wherein the at least one click element and the first and second detent elements of the click device are arranged relative to each other so that upon snap-in of the click device the click element first engages in the one of the two detent elements, before the click element is engageable in the other one of the two detent elements.

18. The infusion tube clamp according to claim 13, wherein one of the two detent elements has an actuating surface for an actuator of an infusion tube clamp actuation of the infusion pump, wherein the second one of the two detent elements is configured to be deflectable further than the first one of the two detent elements.

19. The infusion tube clamp according to claim 13, wherein the infusion tube clamp exhibits in at least one operating state a tube guiding space being radially closed on all sides and having a tube guiding direction arranged transversely to the longitudinal extension of the two clamping legs.

20. The infusion tube clamp according to claim 13, wherein the infusion tube clamp includes an operating flank for an infusion pump locking bar.

21. A method of using an infusion tube clamp at an infusion pump in which a first clamping leg and a second clamping leg of the infusion tube clamp are swiveled toward each other around an infusion tube clamp end area so that an infusion tube can be squeezed, wherein a click device is engaged by at least one click element with a first detachable first detent element into a first snap-in position in which the infusion tube is retained between the two clamping legs, and the click device is engaged by the at least one click element or by another click element with a non-detachable second detent element into a second snap-in position in which the infusion tube is squeezed wherein the non-detachable detent element can be passed by the at least one click element only once in a first direction and the detachable detent element can be passed by the at least one click element both in the first direction and in a second direction opposed to the first direction.

22. The method according to claim 21, wherein for re-opening the infusion tube the second detent element is deflected so that the click device returns from the second snap-in position to the first snap-in position.

23. An infusion pump including a seat for an infusion tube clamp arranged on a tube side and an actuator for actuating the tube-side infusion tube clamp, comprising:
an infusion tube clamp according to claim 13.
